(19) United States
(12) Patent Application Publication
(10) Pub. No.: US 2015/0201996 A1
(43) Pub. Date: Jul. 23, 2015

(54) ELECTROPORATION CONTROLLED WITH REAL TIME IMAGING

(71) Applicants: THE REGENTS OF THE UNIVERSITY OF CALIFORNIA, Oakland, CA (US); ONCOBIONIC, Coto de Caza, CA (US)

(72) Inventors: BORIS RUBINSKY, El Cerrito, CA (US); PAUL MIKUS, Menlo Park, CA (US); GARY ONIK, Berkeley, CA (US)

(21) Appl. No.: 14/627,809

(22) Filed: Feb. 20, 2015

Related U.S. Application Data
(63) Continuation of application No. 11/375,600, filed on Mar. 13, 2006, now abandoned.
(60) Provisional application No. 60/675,695, filed on Apr. 27, 2005.

(51) Int. Cl. A61B 18/14; A61B 8/00

(52) U.S. Cl. CPC A61B 18/1477; A61B 8/4416; A61B 2018/00613

(57) ABSTRACT

A method and a system for producing the method are disclosed whereby irreversible electroporation pulses are produced across an area of target tissue. A medical imaging device is used to create an image of the irreversible electroporation in real time thereby making it possible to determine the area of electroporation and the extent of results obtained and to adjust the positioning of electrodes and/or the current as needed based on the image being viewed.

### CROSS-REFERENCE

This application is a continuation of U.S. patent application Ser. No. 11/375,600, filed Mar. 13, 2006 (now abandoned), which claims the benefit of U.S. Provisional Application No. 60/675,695, filed Apr. 27, 2005, which application is incorporated herein by reference.

### FIELD OF THE INVENTION

This invention relates to the field of electroporation of tissue and specifically to the use of medical imaging technologies applied in real time in order to monitor and control electroporation.

### BACKGROUND OF THE INVENTION

Electroporation is defined as the phenomenon that makes cell membranes permeable by exposing them to certain electric pulses (Weaver, J. C. and Y. A. Chizmadzhev, Theory of electroporation: a review. Bioelectrochem. Bioenerg., 1996. 41: p. 135-60). The permeabilization of the membrane can be reversible or irreversible as a function of the electrical parameters used. In reversible electroporation the cell membrane reseals a certain time after the pulses cease and the cell survives. In irreversible electroporation the cell membrane does not reseal and the cell lyses. (Dev, S. B., Rabussay, D. P., Widera, G., Hofmann, G. A., Medical applications of electroporation, IEEE Transactions of Plasma Science, Vol 28 No 1, February 2000, pp 206-223)

Dielectric breakdown of the cell membrane due to an induced electric field, irreversible electroporation, was first observed in the early 1970s (Neumann, E. and K. Rosenheck, Permeability changes induced by electric impulses in vesicular membranes. J. Membrane Biol., 1972. 10: p. 279-290; Crowley, J. M., Electrical breakdown of biomolecular lipid membranes as an electromechanical instability. Biophysical Journal, 1973. 13: p. 711-724; Zimmermann, U., J. Vienken, and G. Pilwat, Dielectric breakdown of cell membranes, Biophysical Journal, 1974. 14(11): p. 881-899). The ability of the membrane to reseal, reversible electroporation, was discovered separately during the late 1970s (Kinosita Jr, K. and T. Y. Tsong, Hemolysis of human erythrocytes by a transient electric field. Proc. Natl. Acad. Sci. USA, 1977. 74(5): p. 1923-1927; Baker, P. F. and D. E. Knight, Calcium-dependent exocytosis in bovine adrenal medullary cells with leaky plasma membranes. Nature, 1978. 276: p. 620-622; Gauger, B. and F. W. Bentrup, A Study of Dielectric Membrane Breakdown in the Fucus Egg, J. Membrane Biol., 1979. 48(3): p. 249-264).

The mechanism of electroporation is not yet fully understood. It is thought that the electrical field changes the electrochemical potential around a cell membrane and induces instabilities in the polarized cell membrane lipid bilayer. The unstable membrane then alters its shape forming aqueous pathways that possibly are nano-scale pores through the membrane, hence the term “electroporation” (Chang, D. C., et al., Guide to Electroporation and Electrofusion. 1992, San Diego, Calif.: Academic Press, Inc.). Mass transfer can now occur through these channels under electrochemical control. Whatever the mechanism through which the cell membrane becomes permeabilized, electroporation has become an important method for enhanced mass transfer across the cell membrane.

The first important application of the cell membrane permeabilizing properties of electroporation is due to Neumann (Neumann, E., et al., Gene transfer into mouse lyoma cells by electroporation in high electric fields. J. EMBO, 1982. 1: p. 841-5). He has shown that by applying reversible electroporation to cells it is possible to sufficiently permeabilize the cell membrane so that genes, which are macromolecules that normally are too large to enter cells, can after electroporation enter the cell. Using reversible electroporation electrical parameters is crucial to the success of the procedure, since the goal of the procedure is to have a viable cell that incorporates the gene.

Following this discovery electroporation became commonly used to reversible permeabilize the cell membrane for various applications in medicine and biotechnology to introduce into cells or to extract from cells chemical species that normally do not pass, or have difficulty passing across the cell membrane, from small molecules such as fluorescent dyes, drugs and radioactive tracers to high molecular weight molecules such as antibodies, enzymes, nucleic acids, HMW dextrans and DNA.

Following work on cells outside the body, reversible electroporation began to be used for permeabilization of cells in tissue. Heller, R., R. Gilbert, and M. J. Jaroszeski, Clinical applications of electrochemotherapy. Advanced drug delivery reviews, 1999. 35: p. 119-129. Tissue electroporation is now becoming an increasingly popular minimally invasive surgical technique for introducing small drugs and macromolecules into cells in specific areas of the body. This technique is accomplished by injecting drugs or macromolecules into the affected area and placing electrodes into or around the targeted tissue to generate reversible permeabilizing electric field in the tissue, thereby introducing the drugs or macromolecules into the cells of the affected area (Mir, L. M., Therapeutic perspectives of in vivo cell electropermeabilization. Bioelectrochemistry, 2001. 53: p. 1-10).

The use of electroporation to ablate undesirable tissue was introduced by Okino and Mohri in 1987 and Mir et al. in 1991. They have recognized that there are drugs for treatment of cancer, such as bleomycin and cys-platinum, which are very effective in ablation of cancer cells but have difficulties penetrating the cell membrane. Furthermore, some of these drugs, such as bleomycin, have the ability to selectively affect cancerous cells which reproduce without affecting normal cells that do not reproduce. Okino and Mori and Mir et al. separately discovered that combining the electric pulses with an impermeant anticancer drug greatly enhanced the effectiveness of the treatment with that drug (Okino, M. and H. Mohri, Effects of a high-voltage electrical impulse and an anticancer drug on in vivo growing tumors. Japanese Journal of Cancer Research, 1987. 78(12): p. 1319-21; Mir, L. M., et al., Electrochemotherapy potentiation of antitumour effect of bleomycin by local electric pulses. European Journal of Cancer, 1991. 27: p. 68-72). Mir et al. soon followed with clinical trials that have shown promising results and coined the treatment electrochemotherapy (Mir, L. M., et al., Electrochemotherapy, a novel antitumor treatment: first clinical trial. C. R. Acad. Sci., 1991. Ser. III 313(613-8)).

Currently, the primary therapeutic in vivo applications of electroporation are antitumor electrochemotherapy (ECT), which combines a cytotoxic nonpermeant drug with permeabilizing electric pulses and electrogenetherapy (EGT) as a form of non-viral gene therapy, and transdermal drug delivery (Mir, L. M., Therapeutic perspectives of in vivo cell electropermeabilization. Bioelectrochemistry, 2001. 53: p. 1-10). The studies on electrochemotherapy and electrogenetherapy have been recently summarized in several publications (Jaroszeski, M. J., et al., In vivo gene delivery by electroporation. Advanced applications of electrochemistry, 1999. 35: p. 131-137; Heller, R., R. Gilbert, and M. J. Jaroszeski, Clinical applications of electrochemotherapy. Advanced drug delivery reviews, 1999. 35: p. 119-129; Mir, L. M., Therapeutic perspectives of in vivo cell electropermeabilization. Bioelectrochemistry, 2001. 53: p. 1-10; Davalos, R. V., Real Time Imaging for Molecular Medicine through electrical Impedance Tomography of Electroporation, in Mechanical Engineering. 2002, University of California at Berkeley: Berkeley. p. 237). A recent article summarized the results from clinical trials performed in five cancer research centers. Basal cell carcinoma, malignant melanoma, adenocarcinoma and head and neck squamous cell carcinoma were treated for a total of 291 tumors (Mir, L. M., et al., Effective treatment of cutaneous and subcutaneous malignant tumours by electrochemotherapy. British Journal of Cancer, 1998. 77(12): p. 2336-2342).

Electrochemotherapy is a promising minimally invasive surgical technique to locally ablate tissue and treat tumors regardless of their histological type with minimal adverse side effects and a high response rate (Dev, S. B., et al., Medical Applications of Electroporation. IEEE Transactions on Plasma Science, 2000. 28(1): p. 206-223; Heller, R., R. Gilbert, and M. J. Jaroszeski, Clinical applications of electrochemotherapy. Advanced drug delivery reviews, 1999. 35: p. 119-129). Electrochemotherapy, which is performed through the insertion of electrodes into the undesirable tissue, the injection of cytotoxic drugs in the tissue and the application of reversible electroporation parameters, benefits from the ease of application of both high temperature treatment therapies and non-selective chemical therapies and results in outcomes comparable of both high temperature therapies and non-selective chemical therapies.

Irreversible electroporation, the application of electrical pulses which induce irreversible electroporation in cells is also considered for tissue ablation (Davalos, R. V., Real Time Imaging for Molecular Medicine through electrical Impedance Tomography of Electroporation, in Mechanical Engineering. 2002, PhD Thesis, University of California at Berkeley: Berkeley, Davalos, R., L. Mir, Rubinsky B., “Tissue ablation with irreversible electroporation” in print February 2005 Annals of Biomedical Eng,). Irreversible electroporation has the potential for becoming and important minimally invasive surgical technique. However, when used deep in the body, as opposed to the outer surface or in the vicinity of the outer surface of the body, it has a drawback that is typical to all minimally invasive surgical techniques that occur deep in the body, it cannot be closely monitored and controlled. In order for irreversible electroporation to become a routine technique in tissue ablation, it needs to be controllable with immediate feedback. This is necessary to ensure that the targeted areas have been appropriately treated without affecting the surrounding tissue. This invention provides a solution to this problem in the form of medical imaging.

Medical imaging has become an essential aspect of minimally and non-invasive surgery since it was introduced in the early 1980's by the group of Onik and Rubinsky (G. Onik, C. Cooper, H. I. Goldenberg, A. A. Moss, B. Rubinsky, and M. Christianson, “Ultrasonic Characteristics of Frozen Liver,” Cryobiology, 21, pp. 321-328, 1984, J. C. Gilbert, G. M. Onik, W. Haddick, and B. Rubinsky, “The Use of Ultrasound Imaging for Monitoring Cryosurgery,” Proceedings 6th Annual Conference, IEEE Engineering in Medicine and Biology, 107-112, 1984 G. Onik, J. Gilbert, W. K. Haddick, R. A. Filly, P. W. Collen, B. Rubinsky, and L. Farrel, “Sonographic Monitoring of Hepatic Cryosurgery, Experimental Animal Model,” American J. of Roentgenology, May 1985, pp. 1043-1047.) Medical imaging involves the production of a map of various physical properties of tissue, which the imaging technique uses to generate a distribution. For example, in using x-rays a map of the x-ray absorption characteristics of various tissues is produced, in ultrasound a map of the pressure wave reflection characteristics of the tissue is produced, in magnetic resonance imaging a map of proton density is produced, in light imaging a map of either photon scattering or absorption characteristics of tissue is produced, in electrical impedance tomography or induction impedance tomography or microwave tomography a map of electrical impedance is produced.

Minimally invasive surgery involves causing desirable changes in tissue, by minimally invasive means. Often minimally invasive surgery is used for the ablation of certain undesirable tissues by various means. For instance in cryosurgery the undesirable tissue is frozen, in radio-frequency ablation, focused ultrasound, electrical and micro-waves hyperthermia tissue is heated, in alcohol ablation proteins are denaturized, in laser ablation photons are delivered to elevate the energy of electrons. In order for imaging to detect and monitor the effects of minimally invasive surgery, these should produce changes in the physical properties that the imaging technique monitors.

Until our recent studies it was thought that the primary effect of irreversible electroporation in tissue is the production of reversible or irreversible nanoscale pores in the cell membrane. These changes are at the nano-scale and therefore at a scale in which conventional imaging techniques such as ultrasound, CT, MRI, light cannot distinguish differences. The formation of nanopores in the cell membrane has the effect of changing the electrical impedance properties of the cell (Huang, Y, Rubinsky, B., “Micro-electroporation: improving the efficiency and understanding of electrical permeabilization of cells” Biomedical Microdevices, Vo 3, 145-150, 2000. (Discussed in “Nature Biotechnology” Vol 18. pp 368, April 2000), B. Rubinsky, Y Huang. “Controlled electroporation and mass transfer across cell membranes U.S. Pat. No. 6,300,108, Oct. 9, 2001).

Thereafter, electrical impedance tomography was developed, which is an imaging technique that maps the electrical properties of tissue. This concept was proven with experimental and analytical studies (Davalos, R. V., Rubinsky, B., Otten, D. M., “A feasibility study for electrical impedance tomography as a means to monitor tissue electroporation in molecular medicine” IEEE Trans of Biomedical Engineering. Vol. 49, No. 4 pp 400-404, 2002, B. Rubinsky, Y. Huang. “Electrical Impedance Tomography to control electroporation” U.S. Pat. No. 6,387,671, May 14, 2002.)

### SUMMARY OF THE INVENTION

Irreversible electroporation pulses produce an instantaneous and distinct image on conventional medical ultrasound. This distinct image corresponds well with the analytically predicted extent of tissue electroporation and with subsequent histological measurements of tissue ablation with electroporated pulses. The invention is illustrated here with analytical and experimental studies with commercial ultrasound in the pig liver. The present invention shows that conventional ultrasound can be used to monitor and develop controlled treatment planning with irreversible electroporation. Further, the present invention shows that the changes in the imaging characteristics of the electroporated tissue appear almost instantaneously (within a fraction of a minute) as a result of the application of an electrical pulse. This allows for real time monitoring of electroporation and its effects on tissue. Other conventional imaging techniques such as MRI, CT or light imaging can produce similar images when used with irreversible electroporation.

An aspect of the present invention uses conventional imaging with medical ultrasound to produce real time images of the extent of electroporated tissue, starting instantaneously after the application of the pulse.

Another aspect of the invention is a method of controlled tissue ablation whereby irreversible electroporation is monitored and controlled in real time using one or more medical imaging technologies.

Another aspect of the invention comprises placing other types of monitoring devices such as a high impotence needle and/or a thermal couple device in the tissue and monitoring before, during and/or after electroporation which monitoring may be carried out by itself or in combination with the imaging technology described here.

In yet another aspect of the invention test pulses of current are applied which pulses are insufficient to obtain irreversible electroporation and monitoring is carried out during the test pulses and measurements are extrapolated back to determine the amount of voltage, current and duration to obtain the desired degree of electroporation to obtain irreversible electroporation in the targeted tissue.

Yet another aspect of the invention is a method whereby a specific type and area of tissue such as a tumor can be ablated via electroporation while viewed in real time via an imaging methodology such as ultrasound.

These and other aspects of the invention will become apparent to those skilled in the art upon reading this disclosure.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1 is a schematic view of electrodes in place for electroporation of a tumor inside an organ.

FIG. 2 is a schematic view of how electrodes may be placed to limit nerve damage when ablating a tumor.

FIGS. 3A, 3B, 3C and 3D show irreversible electroporated liver tissue.

FIG. 4 shows a schematic of calculated electrical fields in electroporated tissue.

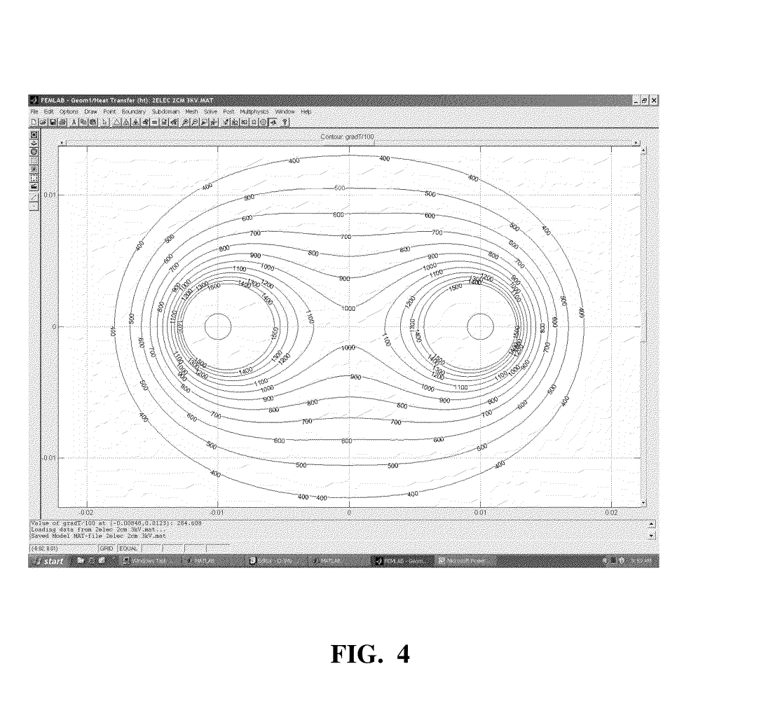

FIGS. 5A, 5B, 5C and 5D show histological images of macroscopic images of electroporated tissue.

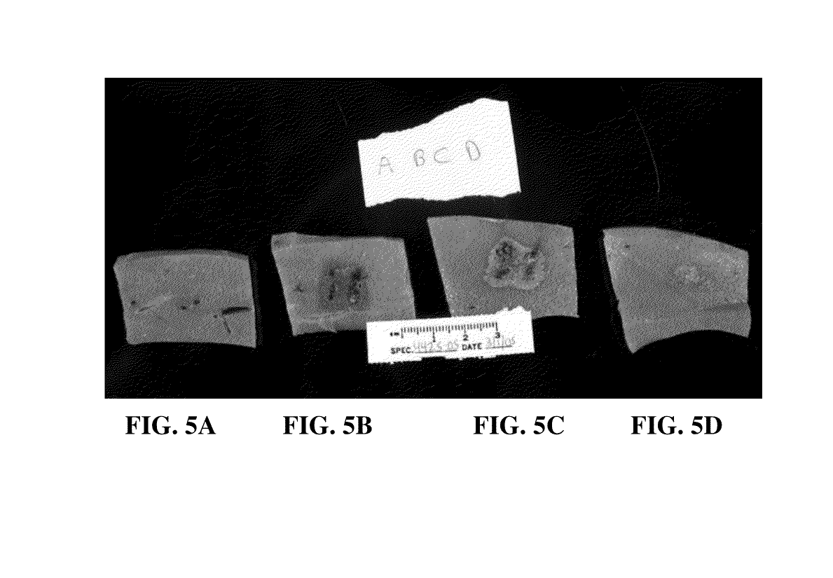

FIG. 6 is a schematic view of an electrode.

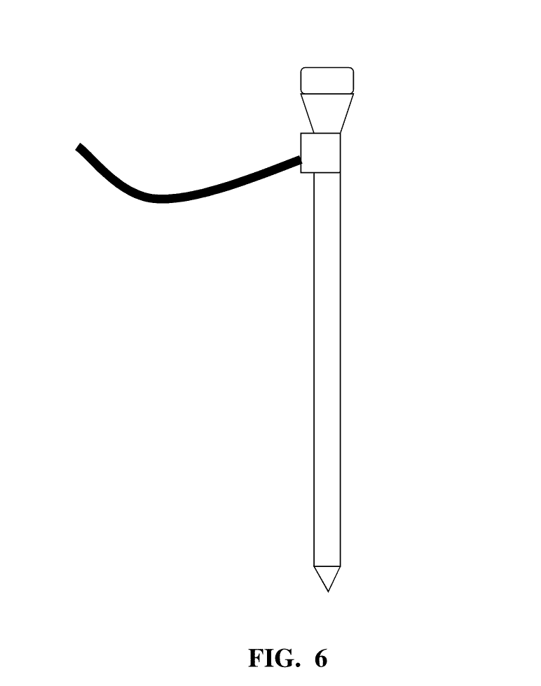

### DETAILED DESCRIPTION OF THE INVENTION

Before the present methods, treatments and devices are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

It must be noted that as used herein and in the appended claims, the singular forms “a”, “an”, and “the” include plural referents unless the context clearly dictates otherwise. Thus, for example, reference to “a pulse” includes a plurality of such pulses and reference to “the sample” includes reference to one or more samples and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “reversible electroporation” encompasses permeabilization of a cell membrane through the application of electrical pulses across the cell. In “reversible electroporation” the permeabilization of the cell membrane ceases after the application of the pulse and the cell membrane permeability reverts to normal or at least to a level such that the cell is viable. Thus, the cell survives “reversible electroporation.” It may be used as a means for introducing chemicals, DNA, or other materials into cells.

The term “irreversible electroporation” also encompasses the permeabilization of a cell membrane through the application of electrical pulses across the cell. However, in “irreversible electroporation” the permeabilization of the cell membrane does not cease after the application of the pulse and the cell membrane permeability does not revert to normal and as such cell is not viable. Thus, the cell does not survive “irreversible electroporation” and the cell death is caused by the disruption of the cell membrane and not merely by internal perturbation of cellular components. Openings in the cell membrane are created and/or expanded in size resulting in a fatal disruption in the normal controlled flow of material across the cell membrane. The cell membrane is highly specialized in its ability to regulate what leaves and enters the cell. Irreversible electroporation destroys that ability to regulate in a manner such that the cell can not compensate and as such the cell dies.

“Ultrasound” is a method used to image tissue in which pressure waves are sent into the tissue using a piezoelectric crystal. The resulting returning waves caused by tissue reflection are transformed into an image.

“MRI” is an imaging modality that uses the perturbation of hydrogen molecules caused by a radio pulse to create an image.

“CT” is an imaging modality that uses the attenuation of an x-ray beam to create an image.

“Light imaging” is an imaging method in which electromagnetic waves with frequencies in the range of visible to far infrared are send into tissue and the tissue's reflection and/or absorption characteristics are reconstructed.

“Electrical impedance tomography” is an imaging technique in which a tissue's electrical impedance characteristics are reconstructed by applying a current across the tissue and measuring electrical currents and potentials

### INVENTION IN GENERAL

In accordance with the present invention specific imaging technologies used in the field of medicine are used to create images of tissue affected by electroporation pulses. The images are created during the process of carrying out irreversible electroporation and are used to focus the electroporation on tissue such as a tumor to be ablated and to avoid ablating tissue such as nerves. The process of the invention may be carried out by placing electrodes, such as a needle electrode in the imaging path of an imaging device. When the electrodes are activated the image device creates an image of tissue being subjected to electroporation. The effectiveness and extent of the electroporation over a given area of tissue can be determined in real time using the imaging technology.

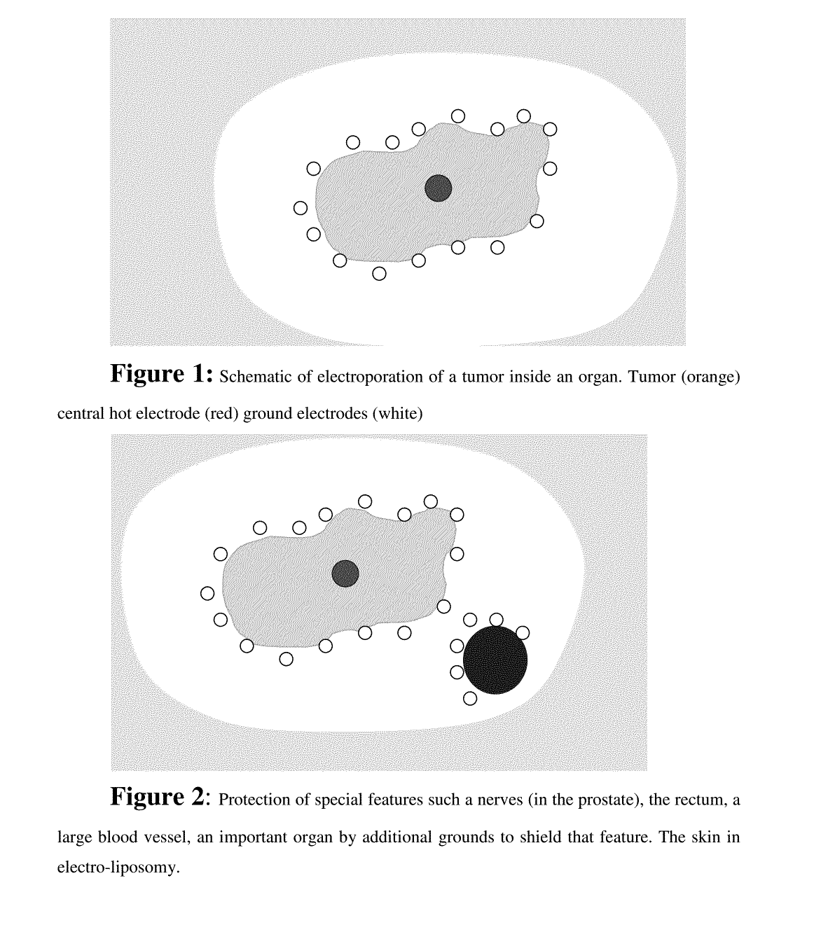

Reversible electroporation requires electrical parameters in a precise range of values that induce only reversible electroporation. To accomplish this precise and relatively narrow range of values (between the onset of electroporation and the onset of irreversible electroporation) when reversible electroporation devices are designed they are designed to generally operate in pairs or in a precisely controlled configuration that allows delivery of these precise pulses limited by certain upper and lower values. In contrast, in irreversible electroporation the limit is more focused on the lower value of the pulse which should be high enough to induce irreversible electroporation. Higher values can be used provided they do not induce burning. Therefore the design principles are such that no matter how many electrodes are use the only constrain is that the electrical parameters between the most distant ones be at least the value of irreversible electroporation. If within the electroporated regions and within electrodes there are higher gradients this does not diminish the effectiveness of the probe. From these principles we can use a very effective design in which any irregular region to be ablated can be treated by surrounding the region with ground electrodes and providing the electrical pulses from a central electrode. The use of the ground electrodes around the treated area has another potential value—it protects the tissue outside the area that is intended to be treated from electrical currents and is an important safety measure. In principle, to further protect an area of tissue from stray currents it would be possible to put two layers of ground electrodes around the area to be ablated. Schematically, the design takes the form shown in a cross section in FIG. 1. It should be emphasized that the electrodes can be infinitely long and can also be curves to better hug the undesirable area to be ablated.

A method is disclosed whereby an electrical pulse or pulses are applied to tissue. The pulses are applied between electrodes and are applied in numbers with currents so as to result in irreversible electroporation of the cells without damaging surrounding cells. Energy waves are emitted from an imaging device such that the energy waves of the imaging device pass through the area positioned between the electrodes and the irreversible electroporation of the cells effects the energy waves of the imaging device in a manner so as to create an image.

Typical values for pulse length for irreversible electroporation are in a range of from about 5 microseconds to about 62,000 milliseconds or about 75 microseconds to about 20,000 milliseconds or about 100 microseconds±10 microseconds. This is significantly longer than the pulse length generally used in intracellular (nano-seconds) electro-manipulation which is 1 microsecond or less—see published U.S. application 2002/0010491 published Jan. 24, 2002. Pulse lengths can be adjusted based on the real time imaging.

The pulse is at voltage of about 100 V/cm to 7,000 V/cm or 200 V/cm to 2000 V/cm or 300V/cm to 1000 V/cm about 600 V/cm±10% for irreversible electroporation. This is substantially lower than that used for intracellular electro-manipulation which is about 10,000 V/cm, see U.S. application 2002/0010491 published Jan. 24, 2002. The voltage can be adjusted alone or with the pulse length based on real time imaging information.

The voltage expressed above is the voltage gradient (voltage per centimeter). The electrodes may be different shapes and sizes and be positioned at different distances from each other. The shape may be circular, oval, square, rectangular or irregular etc. The distance of one electrode to another may be 0.5 to 10 cm., 1 to 5 cm., or 2-3 cm. The electrode may have a surface area of 0.1-5 sq. cm. or 1-2 sq. cm.

The size, shape and distances of the electrodes can vary and such can change the voltage and pulse duration used and can be adjusted based on imaging information. Those skilled in the art will adjust the parameters in accordance with this disclosure and imaging to obtain the desired degree of electroporation and avoid thermal damage to surrounding cells as perceivedin the images.

Thermal effects require electrical pulses that are substantially longer from those used in irreversible electroporation (Davalos, R. V., B. Rubinsky, and L. M. Mir, Theoretical analysis of the thermal effects during in vivo tissue electroporation. Bioelectrochemistry, 2003. Vol 61(1-2): p. 99-107). When using irreversible electroporation for tissue ablation, there may be concern that the irreversible electroporation pulses will be as large as to cause thermal damaging effects to the surrounding tissue and the extent of the tissue ablated by irreversible electroporation will not be significant relative to that ablated by thermal effects. Under such circumstances irreversible electroporation could not be considered as an effective tissue ablation modality as it will act in superposition with thermal ablation. To a degree, this problem is addressed via the present invention using imaging technology.

In one aspect of the invention the imaging device is any medical imaging device including ultrasound, X-ray technologies, magnetic resonance imaging (MRI), light imaging, electrical impedance tomography, electrical induction impedance tomography and microwave tomography. It is possible to use combinations of different imaging technologies at different points in the process. For example, one type of imaging technology can be used to precisely locate a tumor, a second type of imaging technology can be used to confirm the placement of electrodes relative to the tumor. And yet another type of imaging technology could be used to create images of the currents of irreversible electroporation in real time. Thus, for example, MRI technology could be used to precisely locate a tumor. Electrodes could be placed and identified as being well positioned using X-ray imaging technologies. Current could be applied to carry out irreversible electroporation while using ultrasound technology to determine the extent of tissue effected by the electroporation pulses. It has been found that within the resolution of calculations and imaging the extent of the image created on ultrasound corresponds to an area calculated to be irreversibly electroporated. Within the resolution of histology the image created by the ultrasound image corresponds to the extent of tissue ablated as examined histologically.

Because the effectiveness of the irreversible electroporation can be immediately verified with the imaging it is possible to limit the amount of unwanted damage to surrounding tissues and limit the amount of electroporation that is carried out. Further, by using the imaging technology it is possible to reposition the electrodes during the process. The electrode repositioning may be carried out once, twice or a plurality of times as needed in order to obtain the desired degree of irreversible electroporation on the desired tissue such as a tumor.

In accordance with the invention a method may be carried out which comprises several steps. In a first step an area of tissue to be treated by irreversible electroporation is imaged. Electrodes are then placed in the tissue with the tissue to be ablated being positioned between the electrodes. Imaging can also be carried out at this point to confirm that the electrodes are properly placed and the imaging may be used before, during and/or after placement to ensure placement at a desired location. After the electrodes are properly placed pulses of current are run between the two electrodes and the pulsing current is designed so as to minimize damage to surrounding tissue and achieve the desired irreversible electroporation of the target tissue such as a tumor. While the irreversible electroporation is being carried out imaging technology is used and that imaging technology images the irreversible electroporation occurring in real time. While this is occurring the amount of current and number of pulses may be adjusted so as to achieve the desired degree of electroporation. Further, one or more of the electrodes may be repositioned so as to make it possible to target the irreversible electroporation and ablate the desired target tissue.

As described above the invention can be carried out using a wide range of imaging devices. Although the examples below specify the use of ultrasound technology it is possible to use other conventional or other newly developed medical imaging devices which operate using technologies such as CT, MRI or light. Any of these technologies can be used alone or in combination with another imaging technology. Further, these imaging technologies can be used in accordance with the invention to obtain desirable results by themselves. In another aspect of the invention these technologies can be used in combination with other monitoring devices. Alternatively, such other monitoring devices such as the use of thermocouples or a high impedance needle can be used to monitor an area of targeted tissue in accordance with the methodology as described further below.

Thermal ablation methods, particularly cryosurgery, often rely on measurements of a thermocouple placed into the tissue at a critical area to a prevent complications, (by preventing unwanted freezing of tissue) and to confirm the adequacy of the ablation (by reaching a known target temperature that ensures tissue destruction). The monitoring by remote thermocouple is allowed due to the slow nature at which the ablation proceeds allowing modulation of the ablation process based on the feedback from the thermocouple.

Irreversible electroporation has an inherent disadvantage due to the speed at which it occurs. Predictive models of a proposed ablation while accurate in the ideal still do not take into account differences in tissue in homogeneity and needle placements. Due to this speed of ablation, modulation of the ablation process to prevent complications or assess for the adequacy of tissue destruction in critical locations is not possible prior to the full ablation.

In accordance with another aspect of the invention a high impedance needle (to prevent preferential current flow to the monitoring needle) monitoring device is placed into the tissue at a desired location (similar in concept and positioning as would be placed a thermocouple as in a thermal monitoring). Prior to the full electroporation pulse being delivered a “test pulse” is delivered which pulse is a fraction of the proposed full electroporation pulse. This test pulse is in a range that does not cause irreversible electroporation. The monitoring electrode measures the test voltage at the remote location. The voltage measured is then extrapolated back to what would be seen by the monitoring electrode during the full pulse (multiplying by 10 if the test pulse is 10% of the full pulse, since the relationship is linear). If monitoring for a potential complication at the location, a voltage extrapolation that falls under the known level of irreversible electroporation would indicate that the site at which monitoring is taking place is safe. If monitoring at that location for adequacy of electroporation the extrapolation would have to fall above the known level of voltage adequate for irreversible tissue electroporation.

Based on the above it can be seen that one aspect of the invention comprises (a) identifying a target tissue area, (b) placing a monitoring device such as a high impedance needle into the tissue in the area of the identified target tissue, (c) placing electrodes in a manner such that the identified target tissue area is positioned between the electrodes, (d) applying a test current which test current is insufficient to cause irreversible electroporation, (e) monitoring the test current at a remote location, (f) extrapolating back based on the amount of the test current to determine the amount of current necessary to achieve irreversible electroporation, and (g) applying current so as to obtain irreversible electroporation.

In accordance with the method the test current is a fraction of the current necessary in order to obtain irreversible electroporation. Those skilled in the art will adjust the test current as needed. For example, it is possible for the test current to be the full current divided by some integer greater than 1. Thus, the integer can be 10 so that the test current is one tenth of the full current needed to obtain irreversible electroporation. Then, by extrapolating back the amount of current needed for a full pulse can be determined as ten times the test current in that there is a linear relationship.

### EXAMPLE

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

An experimental study with analytical components was performed on a pig liver. The study was conducted in accordance with Good Laboratory Practice regulations as set forth by the 21 Code of Federal Regulations (CFR) Part 58. Full QA oversight, GLP documentation, and a GLP report has provided for this study which fulfills the requirements for submission to federal and/or other agencies requesting non-clinical GLP documentation. The study was performed at Covance Research Products, Berkeley Calif.

Five 100 lb pigs were used in this study. In a typical procedure the pig was anesthetized using general anesthesia. The was liver exposed by an open laparotomy incision. Between two and nine electrode needles were introduced in the liver at desired location under ultrasound monitoring. Approximately 20 different experiments with a variety of needle configuration placements and electroporation potentials were used with the goal of correlating electrical potentials, medical imaging, treatment planning and tissue ablation. The example of electroporation described here used a four needle configuration that is illustrative of all the studies. In this particular experiment four 1 mm needles were placed at 1.5 cm square configuration. The needles were placed under ultrasound monitoring using a template that held the needles in a fixed relationship. Electrical pulses of 2.5 kV were applied eight times for 100 microseconds at 1 Hz in a sequence between each two adjacent needles for a total of four applications. Within a fraction of a minute from the application of the pulses the area that was electroporated was imaged with ultrasound.

Images created are shown in FIG. 1, immediately after the electroporation and 10 minutes after. The images appear hypoechoic compared to the surrounding unelectroporated liver. Four hyperechoic punctuate areas can be seen within the hypoechoic area which indicate the locations of the electroporation needles. Interestingly, over the ensuing hour the hypoechoic region gradually turns hyperechoic, until 1 day later the lesion is uniformly hyperechoic in relation to the unelectroporated liver.

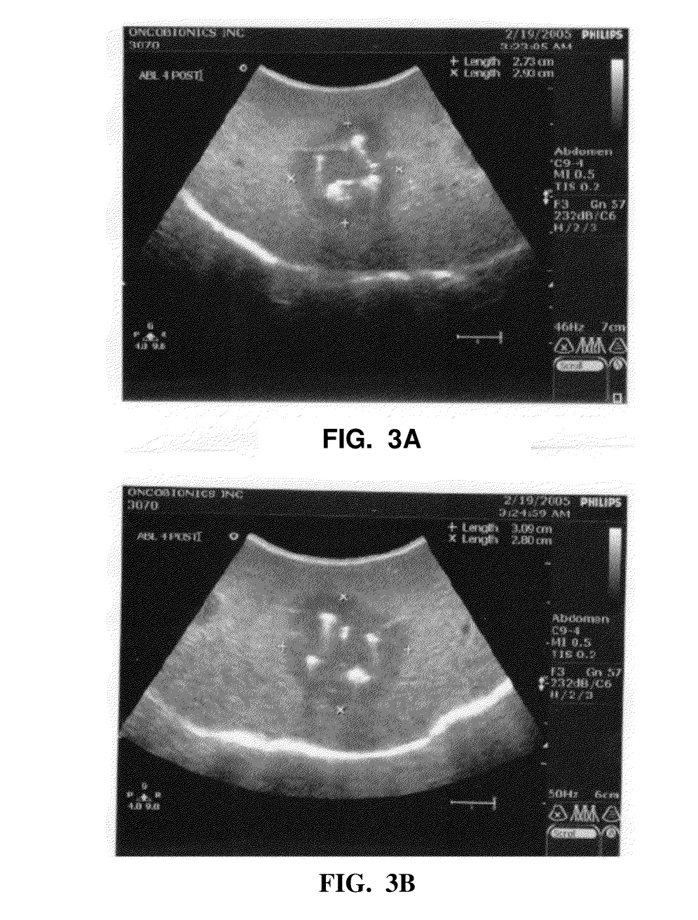

FIG. 2 shows the calculated electrical gradients in the electroporated liver. A comparison with the ultrasound shows that the image of tissue that has been modified by the electroporation pulse corresponds roughly to the extent of irreversible electroporation gradients.

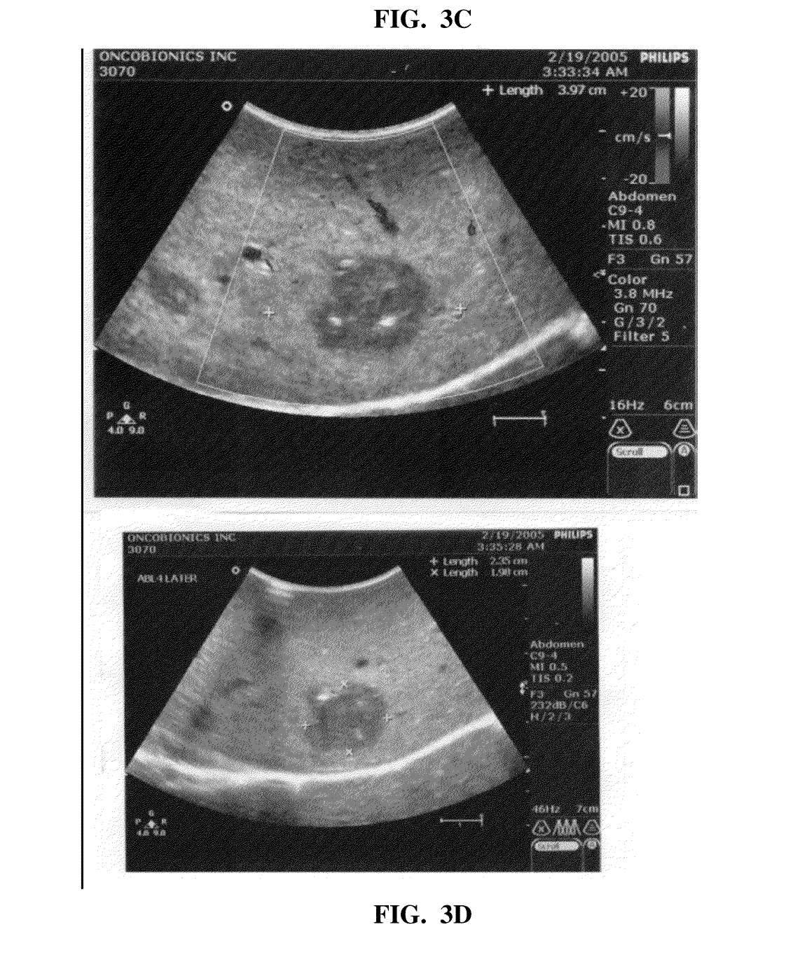

Similarly, FIG. 3 shows a histological macroscopic section of the electroporated region. It corresponds well with the ultrasound image of electroporation.

### Probe Specifications for Irreversible Electroporation

The specifications for the IRE probe are driven by the need to be of a length that will cover the depth needed to reach even the deep complex approaches to the posterior right lobe of the liver, and provide a diameter that will be psychologically acceptable to radiologists to place percutaneously, while causing minimal chance of damage if misplaced. Further, the probe is configured to be usable in a CT scanner, and lastly designed to accommodate injection of a hemostatic agent as it is being withdrawn.

### Probe Specs.

1) Probe width—18 gauge or smaller
2) Active probe length—15 cm
3) Configuration—cable right angle to probe
4) Central diamond removable pointed trocar with Leur lock hub.
5) Variable length insulation.

The back bone of the probe can essentially be an 18 gauge needle of approximately 17 cm long bought from any number of vendors. A potential problem is the interface of the insulation with the probe at its distal end. The transition has to be very smooth to prevent difficulty in placing the probe through the tissue.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1.-16. (canceled)

17. A method of performing irreversible electroporation, comprising:
(a) identifying a target tissue area in a patient;
(b) placing electrodes in the identified target tissue in a manner such that at least a portion of the identified target tissue area is positioned between the electrodes;
(c) applying a test current which test current is insufficient to cause irreversible electroporation;
(d) monitoring the test current;
(e) extrapolating back based on the amount of the test current to determine an amount of current necessary to achieve irreversible electroporation of tissue in the identified target tissue area; and
(f) applying current so as to obtain irreversible electroporation.

18. The method of claim 17, further comprising:
(g) placing a monitoring device into tissue surrounding the identified target tissue, wherein the monitoring device is a high impedance needle.

19. The method of claim 17, further comprising:
(h) imaging the identified target tissue area to determine a degree of electroporation;
(i) adjusting applied current in accordance with the imaging of (h) to achieve irreversible electroporation of tissue in the identified target area.

20. The method of claim 17, wherein the identified target tissue area comprises cancer cells.

21. The method of claim 18, further comprising:
creating a first image of the target tissue area prior to applying current (f);
creating in real-time a second image of the target tissue area after the applying current (f), wherein the second image is an x-ray image, and wherein a location and extent of tissue non-thermally ablated by the irreversible electroporation of cells in the area of the target tissue is adjusted to avoid thermal damage in real-time during an irreversible electroporation procedure by viewing the second image; and
changing at least one parameter or repositioning at least one of the electrodes based on the second image.

22. The method of claim 17, wherein the step of applying current (f) is applied in a plurality of electrical pulses with each pulse having a duration of 5 microseconds to 62 seconds.

23. The method of claim 17, wherein the step of applying current (f) is applied in a plurality of electrical pulses with each pulse having a duration of 5 microseconds to 62 seconds and a voltage in a range of 100 volts/cm to 7000 volts/cm.

24. A method of performing irreversible electroporation, comprising:
(a) identifying a target tissue area in a patient;
(b) placing electrodes in the identified target tissue in a manner such that at least a portion of the identified target tissue area is positioned between the electrodes;
(c) applying a test current which test current is insufficient to cause irreversible electroporation;
(d) monitoring the test current;
(e) extrapolating back based on the amount of the test current to determine an amount of current necessary to achieve irreversible electroporation of tissue in the identified target tissue area; and
(f) applying current based on extrapolating back in (e) so as to obtain irreversible electroporation and avoid thermal damage to tissue surrounding the target tissue.

25. The method of claim 24, further comprising:
(g) placing a monitoring device into tissue surrounding the identified target tissue, wherein the monitoring device is a high impedance needle.

26. The method of claim 25, further comprising:
(h) imaging the identified target tissue area to determine a degree of electroporation;
(i) adjusting applied current with consideration to the imaging of (h) to achieve additional irreversible electroporation of tissue in the identified target area.

27. The method of claim 26, wherein the identified target tissue area comprises cancer cells.

28. The method of claim 25, further comprising:
creating a first image of the target tissue area prior to applying current (f);
creating in real-time a second image of the target tissue area after the applying current (f), wherein the second image is an x-ray image, and wherein a location and extent of tissue non-thermally ablated by the irreversible electroporation of cells in the target tissue area is adjusted to avoid thermal damage in real-time during an irreversible electroporation procedure by viewing the second image; and
changing at least one parameter or repositioning at least one of the electrodes based on the second image.

29. The method of claim 24, wherein the step of applying current (f) is applied in a plurality of electrical pulses with each pulse having a duration of 5 microseconds to 62 seconds.

30. The method of claim 24, wherein the step of applying current (f) is applied in a plurality of electrical pulses with each pulse having a duration of 5 microseconds to 62 seconds and a voltage in a range of 100 volts/cm to 7000 volts/cm.
